(19) United States
(12) Patent Application Publication
(10) Pub. No.: US 2007/0060884 A1
(43) Pub. Date: Mar. 15, 2007

(54) Apparatus for insertion between a medical tube and a body tissue opening

(75) Inventor: Salim Hayek, Cleveland Heights, OH (US)

Correspondence Address: TAROLLI, SUNDHEIM, COVELL & TUMMINO L.L.P., 1300 EAST NINTH STREET, SUITE 1700, CLEVEVLAND, OH, 44114, US

(73) Assignee: The Cleveland Clinic Foundation

(21) Appl. No.: 11/520,391

(22) Filed: Sep. 13, 2006

Related U.S. Application Data
(60) Provisional application No. 60/716,398, filed on Sep. 13, 2005.

(51) Int. Cl. A61M 29/00

(52) U.S. Cl. 604/104

(57) ABSTRACT

An apparatus for insertion between a medical tube and a body tissue opening includes a balloon having inner and outer balloon surfaces separated circumferentially by top and bottom balloon surfaces. The top and bottom balloon surfaces are spaced apart along a longitudinal axis. The inner balloon surface defines a longitudinally extending balloon lumen adapted to receive the medical tube. The outer balloon surface is adapted to selectively contact the body tissue opening. Balloon inflation means are operable to selectively inflate the balloon. The balloon exerts positive pressure on the body tissue opening in response to at least partial inflation. A method of use of the apparatus is also described.

### RELATED APPLICATION

This application claims priority from U.S. Provisional Application No. 60/716,398, filed Sep. 13, 2005, the subject matter of which is incorporated herein by reference.

### TECHNICAL FIELD

The present invention relates to an apparatus for insertion between a medical tube and a body tissue opening, and, more particularly, to such an apparatus and a related method for interposing the apparatus to cushion and fill a body tissue opening through which a medical tube is inserted.

### BACKGROUND OF THE INVENTION

Medical tubes are widely used in the field of medicine for the drainage of blood, air, or other fluids from a body cavity or for the introduction of therapeutic agents to the body. A drainage tube generally remains inserted in the patient for an extended time, such as until the rate and quality of evacuated substances indicate that the drain is no longer needed. For example, a chest tube is ordinarily inserted between two ribs through a thoracic incision in the chest and may be positioned such that a distal tip of the chest tube is located at a point of drainage.

One difficulty with standard medical tube usage is that contact between the rigid medical tube wall and the adjacent body tissue, such as skin, nerves, bones, and other tissues, can cause irritation to the body tissue opening throughout the normal extended period of use. As a result, the medical tube may cause considerable patient discomfort, particularly during removal of the tube from the already-sensitive incision. In the case of a chest tube, this problem is exacerbated by the presence of many intercostal nerves, usually present along the length of each rib, which remain highly sensitive and prone to irritation throughout the duration of the chest intubation. Moreover, a rigid-walled medical tube does not mate closely with the often irregularly-shaped body opening, thereby allowing a path for infection to enter the body.

An example of a device for easing patient discomfort from an inserted medical tube is disclosed in U.S. Pat. No. 6,402,735, issued Jun. 11, 2002 to Paul B. Langevin (hereafter referenced as “the '735 patent”). In the '735 patent, a cylindrical collar may be clipped around the chest tube prior to introduction. The collar is designed to be infused with medication, such as an anesthetic, which elutes through ducts or channels in the outer surface to soothe the tissue in contact with the collar. The cylindrical collar is a rigid, cylindrical clamp which clasps around a chest tube and includes a longitudinal slit to secure the collar around the tube.

Although the anesthetic-eluting property of the device disclosed in the '735 patent may alleviate local pain, the cylindrical design and rigid outer surface of the device do not reduce mechanical tissue irritation. Indeed, the device of the '735 patent may actually increase tissue irritation, as the radial grooves used for drug elution and the longitudinal slit result in an irregularly-shaped surface. Movement of the tube within the body tissue opening may therefore cause more pain and tissue irritation than are caused by a smooth-walled chest tube.

Moreover, while several embodiments are suggested in the '735 patent for securing the collar to a desired point along the chest tube, no structure is suggested for securing the collar to the body tissue opening to prevent dislodgement. Without such securement, the collar might be withdrawn from the opening by a tensile force or, conversely, the collar might be forced into the opening (perhaps tearing the opening open) by a compressive force. Finally, the device of the '735 patent does not appear to provide an adequate mechanism to seal or otherwise prevent infection transmission through the body tissue opening.

Accordingly, it is desirable to provide an apparatus for insertion between a medical tube and a body tissue opening which helps prevent body tissue damage and patient discomfort associated with mechanical contact between the medical tube and the body tissue opening.

### SUMMARY OF THE INVENTION

In an embodiment of the present invention, an apparatus for insertion between a medical tube and a body tissue opening is described. The apparatus includes a balloon having inner and outer balloon surfaces separated circumferentially by top and bottom balloon surfaces. The top and bottom balloon surfaces are spaced apart along a longitudinal axis. The inner balloon surface defines a longitudinally extending balloon lumen adapted to receive the medical tube. The outer balloon surface is adapted to selectively contact the body tissue opening. Balloon inflation means are operable to selectively inflate the balloon. The balloon exerts positive pressure on the body tissue opening in response to at least partial inflation.

In an embodiment of the present invention, a method for interposing an apparatus between a medical tube and a body tissue opening is described. A balloon having inner and outer balloon surfaces separated circumferentially by top and bottom balloon surfaces is provided. The top and bottom balloon surfaces are spaced apart along a longitudinal axis. The inner balloon surface defines a longitudinally extending balloon lumen. The medical tube is placed within the balloon lumen. The body tissue opening is selectively contacted with the outer balloon surface. The balloon is inflated with a balloon inflation means to exert positive pressure on the body tissue opening.

### BRIEF DESCRIPTION OF THE DRAWINGS

For a better understanding of the invention, reference may be made to the accompanying drawings, in which:

FIG. 1 is a side view of an embodiment of the present invention;

FIG. 2 is a top view of the embodiment of FIG. 1, taken along the line 2-2 of FIG. 1;

FIG. 3 is a perspective side view of the embodiment of FIG. 1;

FIG. 4 is a perspective side view of the embodiment of FIG. 1;

FIG. 5 is a side view of the embodiment of FIG. 1 in an alternate configuration;

FIG. 6 is a partial side view of the embodiment of FIG. 1;

FIG. 7A is a top view of the embodiment of FIG. 1;

FIG. 7B is a partial perspective cross-sectional view taken along the line 7B-7B of FIG. 7A;

FIG. 8 is a side view of the embodiment of FIG. 1 in an alternate configuration; and

FIG. 9 is a perspective side view of the embodiment of FIG. 1 in an alternate configuration.

### DESCRIPTION OF EMBODIMENTS

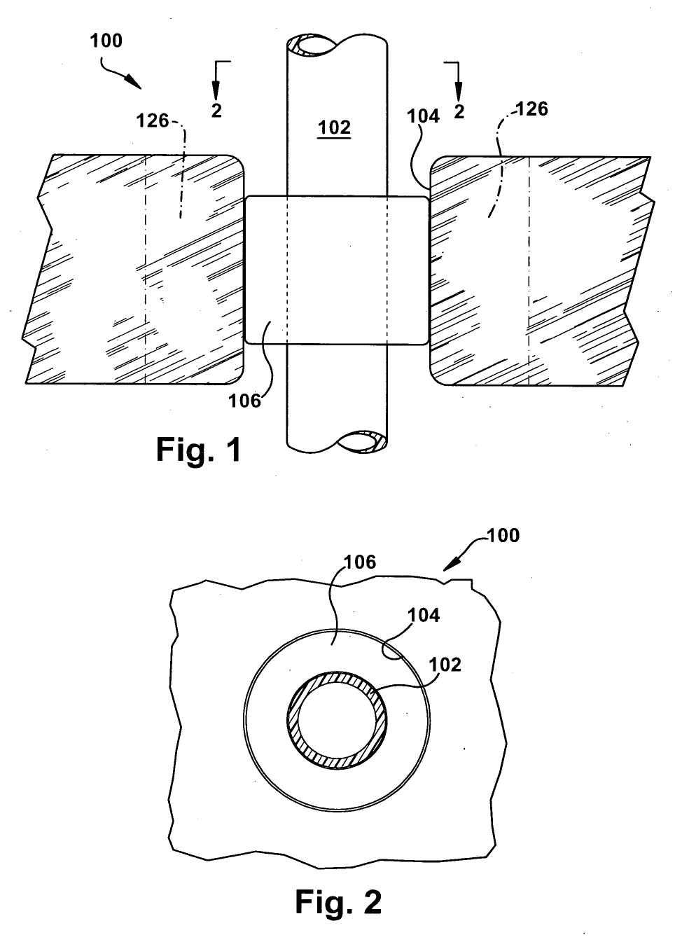

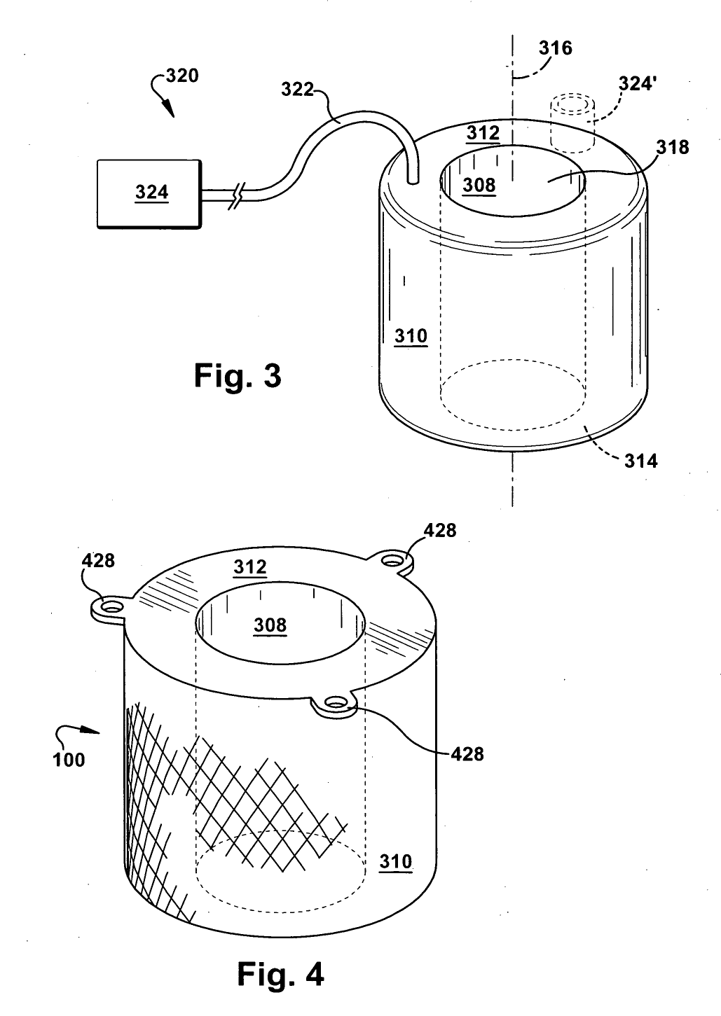

In accordance with the present invention, FIGS. 1 and 2 depict an apparatus 100 for insertion between a medical tube 102 and a body tissue opening 104. “Body tissue opening” may refer to any aperture in a patient's body, natural or created, through which an object such as the medical tube 102 may be at least partially inserted into the body. Nonlimiting examples of body tissue openings 104 include a thoracic incision, a tracheotomy incision, a puncture wound, a vascular access aperture, and the like. The apparatus includes a balloon 106, shown in greater detail in FIG. 3.

The balloon 106 includes inner and outer balloon surfaces 308 and 310, respectively, separated circumferentially by top and bottom balloon surfaces 312 and 314, respectively. The balloon 106 may be of any desired dimensions, material, configuration, and the like, as desired.

The top and bottom balloon surfaces 312 and 314 are spaced apart along a longitudinal axis 316. The top and bottom balloon surfaces 312 and 314 may be distinctly defined, as shown in the Figures for clarity, or may appear to extend contiguously from the inner and/or outer balloon surfaces 308 and 310, as is known in the art. The top and bottom balloon surfaces 312 and 314 may be shaped and sized as needed to circumferentially separate the inner and outer balloon surfaces 308 and 310. The top and bottom balloon surfaces 312 and 314 are optionally used to attach the balloon 106 to the medical tube 102. The precise structures and interactions of the inner, outer, top, and bottom balloon surfaces 308, 310, 312, and 314 are not essential to the present invention and may be readily determined by one of ordinary skill in the art for a particular application. The inner balloon surface 308 defines a longitudinally extending balloon lumen 318 adapted to receive the medical tube 102. The outer balloon surface 310 is adapted to selectively contact the body tissue opening 104.

Balloon inflation means 320 are operable to selectively inflate the balloon 106. The balloon 106 exerts positive pressure on the body tissue opening 104, as shown in FIGS. 1 and 2, in response to at least partial inflation by the balloon inflation means 320. The positive pressure causes the balloon 106 to form-fit to the body tissue opening 104. The amount of pressure may be determined by one of ordinary skill in the art for a particular application of the present invention. For example, the user may wish to exert a relatively low pressure on the body tissue opening 104, so that the balloon 106 provides cushioning for intermittent contact between the medical tube 102 and the body tissue opening. In another example situation, the user may wish to firmly maintain position of the medical tube 102 relative to the body tissue opening 104, perhaps even stretching the body tissue opening slightly, so a relatively high pressure from the balloon 106 to the body tissue opening may be desirable.

The balloon inflation means 320 should place a fluid source (not shown) in fluid communication, and preferably two-way fluid communication, with the balloon lumen 318. The balloon inflation means 320 may include a fill tube 322 extending between the balloon 106 and a remotely located fill valve, shown schematically at 324. Also or instead, the balloon inflation means 320 may include at least one fill valve 324′ located on at least one of the inner, outer, top, and bottom balloon surfaces 308, 310, 312, and 314, as shown in dashed line in FIG. 3. The inflation fluid could be of any suitable type, such as a liquid, gas, or gel and may provide a therapeutic function, to be discussed below, in addition to inflating the balloon 106.

At least one of the outer, top, and bottom balloon surfaces 310, 312, and 314 may include an outer surface feature adapted to engage at least one of the body tissue opening 104 and border tissue (shown at 126 in FIG. 1) adjacent the body tissue opening. The outer surface feature helps to retain the balloon 106 within the body tissue opening 104. Two nonlimiting examples of suitable outer surface features are shown in FIG. 4. The outer balloon surface 310 may have a roughened surface to frictionally engage the body tissue opening 104. Also or instead, the outer surface feature may be an anchor 428 which is attachable to at least one of the body tissue opening 104 and the border tissue 126 adjacent the body tissue opening. For example, the “ears” of FIG. 4 act as anchors 428 adapted for suturing to the border tissue 126. Many other anchor 428 structures could be provided, depending upon the particular application of the apparatus 100, and may readily be designed by one of ordinary skill in the art.

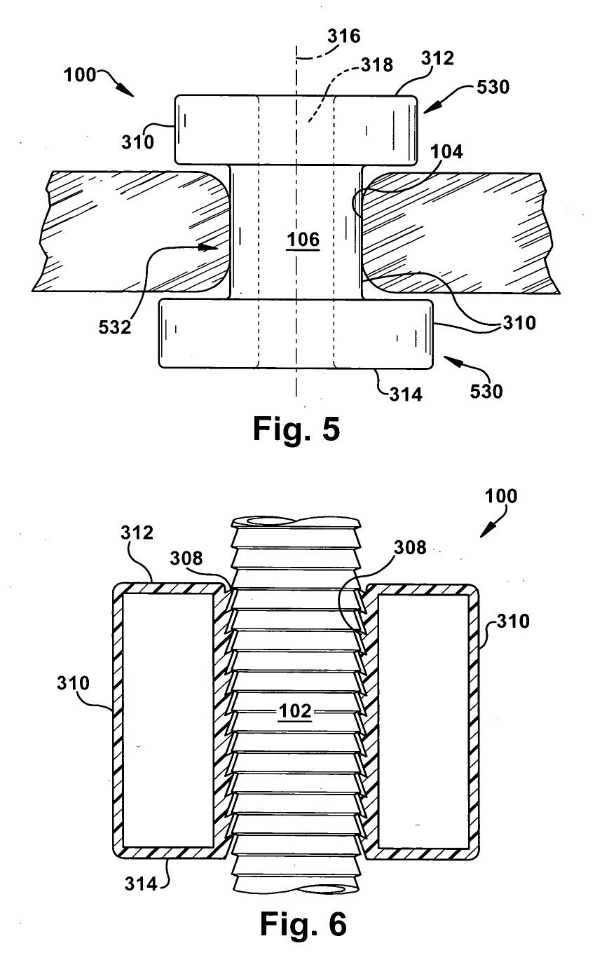

Optionally, the balloon 106 could be shaped to engage the body tissue opening 104 directly, as shown in FIG. 5. The outer balloon surface 310 includes at least one outer surface feature, depicted here as a retention portion 530 (two shown in FIG. 5) and at least one contact portion 532. The contact portion 532 is located radially closer to the longitudinal axis 316 than the retention portion 530 is. The contact portion 532 is adapted to selectively contact the body tissue opening 104. The retention portion 530 is prevented from entering the body tissue opening 104 when the balloon 106 is at least partially inflated. For example, and as shown in FIG. 5, the balloon 106 may have a dumbbell-shaped profile when inflated. The balloon 106 is inserted into the body tissue opening 104 in a deflated state. Once at least partially inflated, the balloon 106 then assumes the shape of FIG. 5 and the retention portions 530 prevent the balloon 106 from being pushed into, or pulled out of, the body tissue opening 104.

Regardless of any engagement between the balloon 106 and the body tissue opening 104 or the border tissue 126, it may be desirable to maintain relative positioning of the balloon 106 and the medical tube 102. The balloon 106 may include an inner surface feature adapted to engage the medical tube 102 to maintain such relative positioning. For example, the inner surface feature may be a portion of a ratcheting mechanism, as shown in FIG. 6. When the inner surface feature includes at least one serration, as in the FIG. 6 ratcheting mechanism, the medical tube 102 may be shaped to engage the inner surface feature as in FIG. 6, or the medical tube may be associated with an engagement structure (not shown). In any case, the inner surface feature need only be adapted to engage the medical tube 102 to maintain such relative positioning and may readily be designed by one of ordinary skill in the art. As an alternative to the use of an inner surface feature to maintain the relative positioning, the balloon 106 and medical tube 102 may be integrally formed.

The apparatus 100 may be adapted to elute a therapeutic agent, such as, but not limited to, an anesthetic, anti-inflammatory, or antiseptic fluid, to a target tissue. The target tissue may be at least one of the body tissue opening 104, a border tissue 126 adjacent the body tissue opening, and any other tissue with which the apparatus 100 may be in contact or in close proximity. “Elute” is used herein to indicate that a therapeutic agent is released, leached, diffused, or otherwise provided to the target tissue.

The therapeutic agent may be eluted using one or more of several available modes of delivery. The balloon 106 may be adapted to elute the therapeutic agent. For example, at least one of the inner, outer, top, and bottom balloon surfaces 308, 310, 312, and 314 may be impregnated with a therapeutic agent adapted to self-release over time or instantaneously, possibly responsive to contact with the target tissue. The therapeutic agent may be provided inside the balloon 106 and released through one or more holes or semipermeable areas (not shown) in at least one of the inner, outer, top, and bottom balloon surfaces 308, 310, 312, and 314. When the therapeutic agent is provided inside the balloon 106, the balloon inflation means 320 may be adapted to introduce the therapeutic agent to the balloon, optionally using the therapeutic agent as an inflation fluid for the balloon.

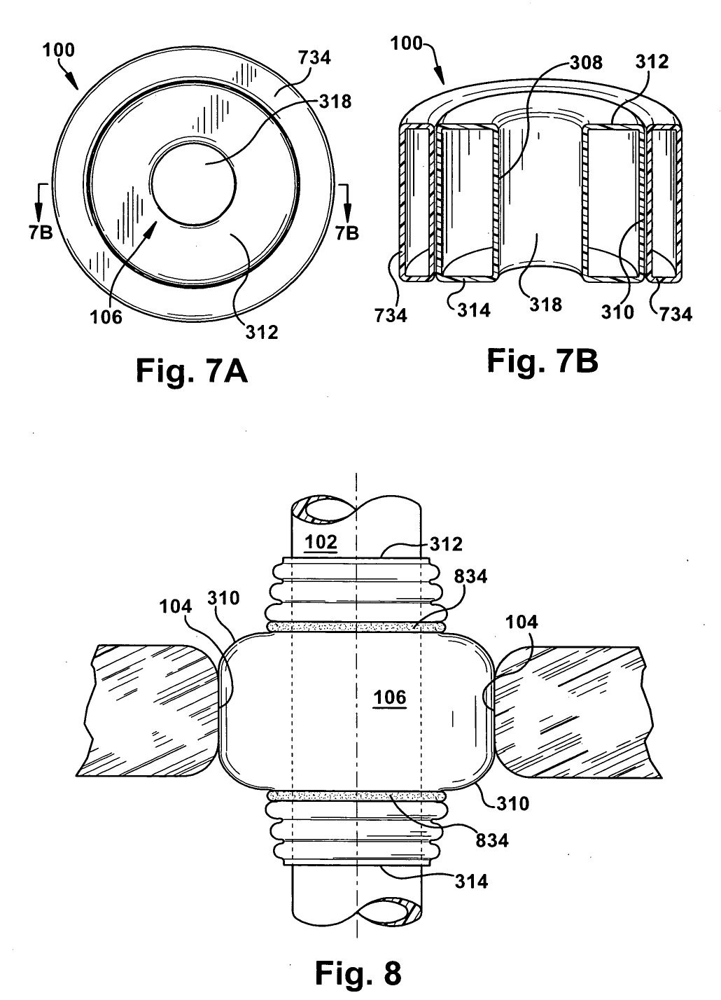

The therapeutic agent, when present, may be provided with the assistance of a therapeutic sleeve 734, shown in the views of FIGS. 7A and 7B. The therapeutic sleeve 734 may surround or cover all or part of at least one of the inner, outer, top, and bottom balloon surfaces 308, 310, 312, and 314. The therapeutic sleeve 734 may have any suitable structure and any suitable degree of attachment to the balloon 106, and may readily be designed for a particular application by one of ordinary skill in the art. The therapeutic sleeve 734 is shown in FIGS. 7A and 7B as an inflatable sleeve (the fill mechanisms are omitted for clarity) surrounding the balloon 106 and having a similar structure to the balloon 106. The therapeutic sleeve 734 could be at least partially impregnated with a therapeutic agent and/or could release a therapeutic agent held therein, as discussed previously with reference to the balloon 106.

The use of a separate therapeutic sleeve 734 with the apparatus 100 may allow the user to select and/or interchange one or more therapeutic agents for a particular application at the time of the surgical procedure. Providing therapeutic agents to the apparatus 100 at the time of use may allow the user to customize the number and type of therapeutic agents used as needed. Therefore, the therapeutic agents may be stored and used as needed, separately from the apparatus 100, which may result in greater freshness of the agents and less expense and effort in providing the apparatus 100.

It may be desirable for the active longitudinal length of the outer balloon surface 310 to be adjustable in response to the longitudinal depth of the body tissue opening 104, thus allowing the apparatus 100 to more closely match the body tissue opening. One optional structure for changing the longitudinal length of the outer balloon surface 310 is shown in FIG. 8 as a balloon constriction means 834. For example, an elastic band, a length of suture thread, a wire clamp, or any other suitable mechanism may be used as a balloon constriction means 834. When the outer balloon surface 310 has a longitudinal length which is greater than desired, at least one balloon constriction means 834 (two shown in FIG. 8) is placed at a desired location along the outer balloon surface. The balloon constriction means 834 holds a portion of the outer balloon surface 310 firmly to the medical tube 102 and thereby prevents inflation fluid from traveling to the entire interior of the balloon 106. The location of the balloon constriction means 834 should be chosen to allow the balloon inflation means 320 to inflate the longitudinal portion of the balloon 106 as desired. As shown in FIG. 8, portions of the balloon 106 located on an opposite side of the balloon constriction means 834 from the body tissue opening 104 may hang slackly adjacent the medical tube 102. These slack portions may be removed, if desired, when the balloon constriction means 834 seals the balloon 106 to the medical tube 102 adequately for the balloon 106 to retain a desired amount of inflation fluid.

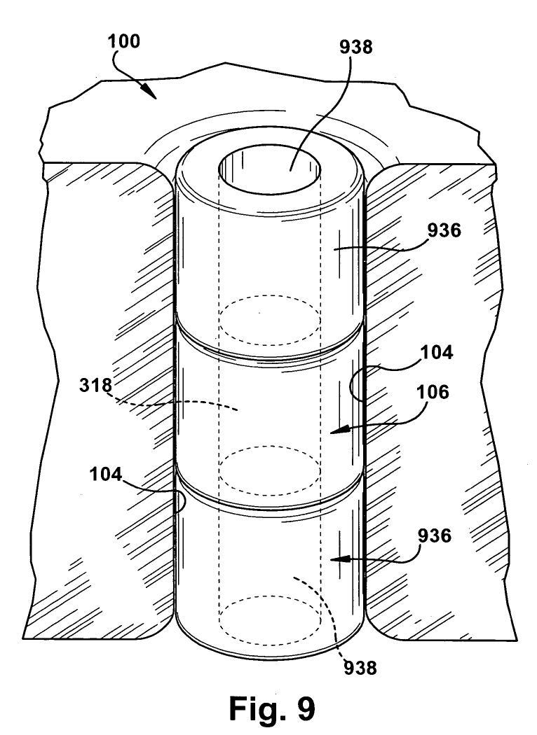

Another structure which may assist in adjusting the longitudinal length of the apparatus 100 is shown in FIG. 9. In the apparatus 100 of FIG. 9, the balloon 106 is a primary balloon 106, and at least one secondary balloon 936 (two shown) is located longitudinally adjacent the primary balloon 106. The secondary balloon 936 defines a secondary balloon lumen 938 adapted to receive the medical tube 102. The primary and secondary balloons 106 and 936 may be selectively inflated in response to a longitudinal depth of the body tissue opening 104. The primary and secondary balloons 106 and 936 may be selectively inflated in any desired manner. For example, the primary and secondary balloons 106 and 936 may each be at least partially inflated by separate balloon inflation means (not shown). The primary and secondary balloons 106 and 936 may be, but are not necessarily, matched in size, shape, material, configuration, or any other property.

In use, the apparatus 100 may be interposed between a medical tube 102 and a body tissue opening 104 to cushion the interface therebetween, to maintain a position of the medical tube within the body tissue opening, and/or to elute one or more therapeutic agents to the body tissue opening or another target tissue.

At least a portion of the balloon 106 is placed within the body tissue opening 104. The balloon 106 may be deflated for placement within the body tissue opening 104 or may be at least partially inflated during placement. The body tissue opening 104 is selectively contacted with the outer balloon surface 310. This contact may occur due to user positioning of the balloon 106 and/or due to at least partial inflation of the balloon 106. Once the balloon 106 is positioned as desired, the balloon is at least partially inflated with the balloon inflation means 320 to exert a positive pressure on the body tissue opening 104. The positive pressure may be chosen to maintain the balloon 106 in position within the body tissue opening 104. Optionally, one or more outer surface features on the outer, top, and/or bottom balloon surfaces 310, 312, and 314 may be provided and engaged to help maintain the position of the balloon 106 with respect to the body tissue opening 104.

Before, during, or after the placement of the balloon 106 within the body tissue opening 104, the medical tube 102 may be placed within the balloon lumen 318. Optionally, the balloon 106 may be formed integrally with the medical tube 102. If desired, one or more inner surface features on the inner balloon surface 308 may be provided and engaged to help maintain the position of the balloon 106 with respect to the medical tube 102.

At any time during use of the apparatus 100, a therapeutic agent may be provided to the body tissue opening 104 or another target tissue, in any suitable manner. Also at any time during use of the apparatus 100, the active longitudinal length of the apparatus may be adjusted through use of one or more balloon constriction means 834 and/or secondary balloons 936.

When the user is ready to remove the medical tube 102 from the patient, the balloon 106 may be at least partially deflated. Such deflation allows the balloon 106 to either release the medical tube 102 from the balloon lumen 318 or to disengage the outer balloon surface 310 from the body tissue opening 104. The medical tube 102 may then be withdrawn from the patient, with or without the balloon 106 attached. Any inner or outer surface features present may be disengaged at an appropriate time to allow release of the medical tube 102 from the patient in a desired manner.

While aspects of the present invention have been particularly shown and described with reference to the preferred embodiment above, it will be understood by those of ordinary skill in the art that various additional embodiments may be contemplated without departing from the spirit and scope of the present invention. For example, the inner surface feature could be a screw thread allowing the medical tube 102 to advance or retract upon rotary motion within the balloon lumen 318. The balloon 106 could have any suitable dimensions (including length). The balloon 106 could be associated with the medical tube 102 at any desired location along the length of the medical tube 102. The balloon 106 could be designed to retain the medical tube 102 in any desired angular or linear relationship with the body tissue opening 104. A pressure valve means could provide a selective fluid connection between the primary and secondary balloons 106 and 936 to allow the secondary balloon to receive inflation fluid responsive to the fluid pressure in the primary balloon exceeding a predetermined threshold. The structures of the apparatus 100 may be made of any suitable rigid or flexible material, using any suitable manufacturing process. Either or both of the inner and outer surface features may be an adhesive. A device or method incorporating any of these features should be understood to fall under the scope of the present invention as determined based upon the claims below and any equivalents thereof.

The method and apparatus of certain embodiments of the present invention, when compared with other apparatus and methods, may have the advantages of helping prevent body tissue damage and patient discomfort associated with mechanical contact between the medical tube and an insertion opening in the body tissue. Such advantages are particularly worthy of incorporating into the design, manufacture, and operation of medical tubes.

Other aspects, objects, and advantages of the present invention can be obtained from a study of the drawings, the disclosure, and the appended claims.

Having described the invention, I claim:

1. An apparatus for insertion between a medical tube and a body tissue opening, the apparatus comprising:
a balloon having inner and outer balloon surfaces separated circumferentially by top and bottom balloon surfaces, the top and bottom balloon surfaces being spaced apart along a longitudinal axis;
the inner balloon surface defining a longitudinally extending balloon lumen adapted to receive the medical tube;
the outer balloon surface being adapted to selectively contact the body tissue opening; and
balloon inflation means operable to selectively inflate the balloon, the balloon exerting positive pressure on the body tissue opening in response to at least partial inflation.

2. The apparatus of claim 1, wherein the balloon inflation means includes a fill tube extending between the balloon and a remotely located fill valve.

3. The apparatus of claim 1, wherein the balloon inflation means includes a fill valve located on at least one of the inner, outer, top, and bottom balloon surfaces.

4. The apparatus of claim 1, wherein the outer balloon surface includes an outer surface feature adapted to engage at least one of the body tissue opening and border tissue adjacent the body tissue opening to retain the balloon within the body tissue opening.

5. The apparatus of claim 4, wherein the outer surface feature is an anchor attachable to at least one of the body tissue opening and border tissue adjacent the body tissue opening.

6. The apparatus of claim 1, wherein the inner balloon surface includes an inner surface feature adapted to engage the medical tube to maintain relative positioning of the balloon and the medical tube.

7. The apparatus of claim 6, wherein the inner surface feature is a portion of a ratcheting mechanism.

8. The apparatus of claim 1, wherein the apparatus is adapted to elute a therapeutic agent to the body tissue opening.

9. The apparatus of claim 8, wherein the balloon is adapted to elute the therapeutic agent.

10. The apparatus of claim 9, wherein the therapeutic agent is introduced to the balloon by the balloon inflation means.

11. The apparatus of claim 8, including a therapeutic sleeve surrounding at least a portion of the outer balloon surface, the therapeutic sleeve being adapted to elute the therapeutic agent.

12. The apparatus of claim 1, wherein the outer balloon surface includes at least one retention portion and at least one contact portion, the contact portion being located radially closer to the longitudinal axis than the retention portion, the contact portion adapted to selectively contact the body tissue opening, and the retention portion prevented from entering the body tissue opening when the balloon is at least partially inflated.

13. The apparatus of claim 1, wherein the balloon is formed integrally with the medical tube.

14. The apparatus of claim 1, including at least one balloon constriction means adapted to change the longitudinal length of the outer balloon surface in response to a longitudinal depth of the body tissue opening.

15. The apparatus of claim 1, wherein the balloon is a primary balloon and at least one secondary balloon is located longitudinally adjacent the primary balloon and defines a secondary balloon lumen adapted to receive the medical tube, the primary and secondary balloons being selectively inflated in response to a longitudinal depth of the body tissue opening.

16. A method for interposing an apparatus between a medical tube and a body tissue opening, the method comprising the steps of:
providing a balloon having inner and outer balloon surfaces separated circumferentially by top and bottom balloon surfaces, the top and bottom balloon surfaces being spaced apart along a longitudinal axis, the inner balloon surface defining a longitudinally extending balloon lumen;
placing the medical tube within the balloon lumen;
selectively contacting the body tissue opening with the outer balloon surface; and
inflating the balloon with a balloon inflation means to exert positive pressure on the body tissue opening.

17. The method of claim 16, wherein the balloon inflation means includes a fill tube extending between the balloon and a remotely located fill valve.

18. The method of claim 16, wherein the balloon inflation means includes a fill valve located on at least one of the inner, outer, top, and bottom balloon surfaces.

19. The method of claim 16, including the steps of:
providing an outer surface feature on the balloon outer balloon surface; and
engaging at least one of the body tissue opening and border tissue adjacent the body tissue opening with the outer surface feature to retain the balloon within the body tissue opening.

20. The method of claim 16, including the steps of:
providing an inner surface feature on the inner balloon surface; and
engaging the medical tube with the inner surface feature to maintain relative positioning of the balloon and the medical tube.

21. The method of claim 16, including the step of providing a therapeutic agent to the body tissue opening.

22. The method of claim 21, wherein the step of providing a therapeutic agent to the body tissue opening includes the step of eluting the therapeutic agent from the balloon.

23. The method of claim 22, including the step of introducing the therapeutic agent to the balloon with the balloon inflation means.

24. The method of claim 21, wherein the step of providing a therapeutic agent to the body tissue opening includes the steps of:
surrounding at least a portion of the outer balloon with a therapeutic sleeve; and
eluting the therapeutic agent from the therapeutic sleeve.

25. The method of claim 16, wherein the outer balloon surface includes at least one retention portion and at least one contact portion, the contact portion being located radially closer to the longitudinal axis than the retention portion, and including the steps of:
selectively contacting the body tissue opening with the contact portion; and
preventing the retention portion from entering the body tissue opening when the balloon is at least partially inflated.

26. The method of claim 16, wherein the balloon is formed integrally with the medical tube.

27. The method of claim 16, including the steps of:
providing at least one balloon constriction means; and
changing the longitudinal length of the outer surface with the balloon constriction means in response to a longitudinal depth of the body tissue opening.

28. The method of claim 16, wherein the balloon is a primary balloon and including the steps of:
providing at least one secondary balloon, located longitudinally adjacent the primary balloon and defining a secondary balloon lumen adapted to receive the medical tube, and
selectively inflating the primary and secondary balloons in response to a longitudinal depth of the body tissue opening.
